(19) United States
(12) Patent Application Publication
(10) Pub. No.: US 2025/0051619 A1
(43) Pub. Date: Feb. 13, 2025

(54) SEMI-CRYSTALLINE POLYOLEFIN CONSTRUCTION ADHESIVE

(71) Applicant: H.B. Fuller Company, St. Paul, MN (US)

(72) Inventors: Daniel A. Everson, Lakeville, MN (US); Ethan A. Gormong, White Bear Lake, MN (US)

(21) Appl. No.: 18/794,665

(22) Filed: Aug. 5, 2024

Related U.S. Application Data
(60) Provisional application No. 63/518,175, filed on Aug. 8, 2023.

(51) Int. Cl. C09J 123/16; A61L 15/20; A61L 15/24; A61L 15/58; C09J 5/06; C09J 11/06; C09J 11/08

(52) U.S. Cl. CPC C09J 123/16; A61L 15/20; A61L 15/24; A61L 15/585; C09J 5/06; C09J 11/06; C09J 11/08

(57) ABSTRACT

The invention includes a hot melt adhesive composition based on polyolefin polymers that can be used for applications (e.g., construction applications) in a disposable absorbent article. The olefin based hot melt adhesive composition has surprisingly good cohesive strength.

### CROSS REFERENCE TO RELATED APPLICATIONS

This application claims the benefit of U.S. Provisional Patent Application No. 63/518,175 filed on Aug. 8, 2023, which is incorporated herein.

### BACKGROUND

Adhesives are often used to bond substrates together to maintain the two substrates in a fixed relation to each other. In the area of industrial adhesives, hot melt adhesives are commonly used to bond together a wide variety of articles including disposable absorbent articles comprising non-woven substrates e.g., adult incontinence products, disposable diapers, sanitary napkins, bed pads, puppy pads, medical dressings, etc.

There can be multiple hot melt adhesives used in the manufacture of a disposable absorbent article. For example, in the manufacture of a disposable diaper, hot melt adhesives are used in construction (e.g., bonding the back sheet to the nonwoven and optionally the absorbent pad), elastic attachment (e.g., bonding the elastic material to the back sheet in for example the leg or waist area), and for core stabilization (e.g., applying an adhesive to the absorbent core to increase the strength of the core).

Hot melt adhesives for construction applications formulated with polyolefin polymers tend to have lower cohesive strength as compared to rubber based products. It would be useful to have olefin based hot melt adhesives that have improved cohesive strength.

### SUMMARY

In one aspect, the invention features a hot melt adhesive composition including a first propylene-based copolymer that has a comonomer content of less than 5% by weight, a Heat of Fusion from 38 joules/gram to 68 joules/gram and a Melt Flow Rate of from 2 grams/10 minute to 40 grams/10 minute when tested at 230° C./2.16 kg according to ASTM D 1238, a second propylene-based copolymer that has a comonomer content of from 5% by weight to 30% by weight, a tackifying agent, and a plasticizer.

In one embodiment, the first propylene-based copolymer has a Heat of Fusion from 40 joules/gram to 65 joules/gram. In another embodiment, the first propylene-based copolymer and the second propylene-based copolymer are metallocene catalyzed. In a different embodiment, the second propylene-based copolymer has a Brookfield Viscosity at 190° C. of no greater than 20,000 cps. In one embodiment, the hot melt adhesive composition includes at least 22% by weight of a plasticizer, or even at least 24% by weight of a plasticizer.

In a different embodiment, the hot melt adhesive composition includes from 2% by weight to 20% by weight of the first propylene-based copolymer, from 2% by weight to 25% by weight of the second propylene-based copolymer, from 30% by weight to 60% by weight of a tackifying agent, and from 22% by weight to 35% by weight of a plasticizer.

In another aspect the invention features a hot melt adhesive composition including a first propylene-based copolymer that has a comonomer content of less than 8% by weight, a Heat of Fusion from 38 joules/gram to 68 joules/gram, and a Melt Flow Rate of from 2 grams/10 minute to 40 grams/10 minute when tested at 230° C./2.16 kg according to ASTM D 1238, a second propylene-based copolymer that has a comonomer content of from 8% by weight to 30% by weight and a Melt Flow Rate of from 2 grams/10 minute to 50 grams/10 minute when tested at 230° C./2.16 kg according to ASTM D 1238, a tackifying agent and a plasticizer.

In one embodiment, the first propylene-based copolymer has a Heat of Fusion from 40 joules/gram to 65 joules/gram. In another embodiment, he hot melt adhesive composition includes from 3% by weight to 12% by weight of the first propylene-based copolymer, from 3% by weight to 12% by weight of the second propylene-based copolymer, from 35% by weight to 60% by weight of a tackifying agent, and from 25% by weight to 35% by weight of a plasticizer.

In one embodiment, the total of the first propylene-based copolymer and the second propylene-based copolymer makes up from 10% by weight to 20% by weight of the hot melt adhesive composition. In a different embodiment, the total of the first propylene-based copolymer and the second propylene-base copolymer makes up from 12% by weight to 18% by weight of the hot melt adhesive composition.

In one embodiment, the first propylene-based copolymer has a melt flow rate of from 2 grams/10 minute to 20 grams/10 minute when tested at 230° C./2.16 kg according to ASTM D 1238. In a different embodiment, the first propylene-based copolymer and second propylene-based copolymer are propylene ethylene copolymers. In another embodiment, the tackifying agent is a hydrogenated hydrocarbon. In one embodiment, the tackifying agent has a Ring & Ball Softening Point of from 90° C. to 110° C.

In another embodiment, the plasticizer is an oil. In another embodiment, the hot melt adhesive composition has a Heat of Fusion of from 6 joules/gram to 20 joules/gram.

In a different embodiment, the invention includes a disposable absorbent article made with one of the inventive hot melt adhesive compositions. In another embodiment, the disposable absorbent article includes a first substrate, a second substrate, and the inventive hot melt adhesive composition, wherein the hot melt adhesive composition is applied to at least one of the first and second substrate.

In one embodiment, the disposable absorbent article is selected from the group consisting of diaper, adult incontinence article and sanitary hygiene article. In another embodiment the inventive hot melt adhesive composition is used in the disposable absorbent article for an application selected from the group consisting of construction and back sheet lamination.

The hot melt adhesive compositions of this invention include a blend of olefin polymers that work together to create physical crosslinks, resulting in semi-crystalline hot melt adhesive compositions that can form strong durable bonds and are particularly useful in the production of disposable absorbent articles.

### Glossary

In reference to the invention, these terms have the meanings set forth below:

Propylene-based refers to a polymer that comprises at least 50% by weight propylene.

### DETAILED DESCRIPTION

### Hot Melt Adhesive Composition

The invention features a hot melt adhesive composition including a first propylene-based copolymer that is single-site catalyzed, has a comonomer content of less than 5% by weight, a Heat of Fusion from 40 joules/gram to 65 joules/gram and a Melt Flow Rate of from 2 grams/10 minute to 40 grams/10 minute when tested at 230° C./2.16 kg according to ASTM D 1238, a second propylene-based copolymer that has a comonomer content of from 5% by weight to 30% by weight, a tackifying agent; and a plasticizer.

The invention features a hot melt adhesive composition including from 4% by weight to 20% by weight of a first propylene-based copolymer that is single-site catalyzed, has a comonomer content of less than 5% by weight, a Heat of Fusion from 40 joules/gram to 65 joules/gram and a Melt Flow Rate of from 2 grams/10 minute to 40 grams/10 minute when tested at 230° C./2.16 kg according to ASTM D 1238, from 4% by weight to 25% by weight of a second propylene-based copolymer that has a comonomer content of from 5% by weight to 30% by weight, from 30% by weight to 60% by weight of a tackifying agent and from 22% by weight to 35% by weight of a plasticizer.

The invention further features a hot melt adhesive composition including a first propylene-based copolymer that is single-site catalyzed and has a comonomer content of less than 8% by weight, a Heat of Fusion from 40 joules/gram to 65 joules/gram, and a Melt Flow Rate of from 2 grams/10 minute to 40 grams/10 minute when tested at 230° C./2.16 kg according to ASTM D 1238, a second propylene-based copolymer that is single-site catalyzed and has a comonomer content of from 8% by weight to 30% by weight, having a Melt Flow Rate of from 2 grams/10minute to 50 grams/10 minute when tested at 230° C./2.16 kg according to ASTM D 1238, a tackifying agent; and a plasticizer.

The invention further features a hot melt adhesive composition including from 3% by weight to 12% by weight of a first propylene-based copolymer that is single-site catalyzed and has a comonomer content of less than 8% by weight, a Heat of Fusion from 40 joules/gram to 65 joules/gram, and a Melt Flow Rate of from 2 grams/10 minute to 40 grams/10 minute when tested at 230° C./2.16 kg according to ASTM D 1238, from 3% by weight to 12% by weight of a second propylene-based copolymer that is single-site catalyzed and has a comonomer content of from 8% by weight to 30% by weight, having a Melt Flow Rate of from 2 grams/10minute to 50 grams/10 minute when tested at 230° C./2.16 kg according to ASTM D 1238, from 35% by weight to 60% by weight of a tackifying agent and from 25% by weight to 35% by weight of a plasticizer.

The invention also features a hot melt adhesive composition including a first propylene-based copolymer that is single-site catalyzed and has a comonomer content of less than 8% by weight, a Heat of Fusion from 40 joules/gram to 65 joules/gram and a Melt Flow Rate of from 2 grams/10 minute to 40 grams/10 minute when tested at 230° C./2.16 kg according to ASTM D 1238, a second propylene-based copolymer that is single-site catalyzed and has a comonomer content of from 8% by weight to 30% by weight, having a Melt Flow Rate of from 2 grams/10minute to 50 grams/10 minute when tested at 230° C./2.16 kg according to ASTM D 1238, a tackifying agent; and a plasticizer wherein the first propylene-based copolymer and the second propylene-based copolymer include 10% by weight to 20% by weight of the hot melt adhesive composition.

The first propylene-based copolymer is highly crystalline and high in molecular weight. This results in a hot melt adhesive composition that is semi-crystalline. The inventors theorize that when crystallinity results from the polymer rather than wax, improved cohesion of the hot melt adhesive as witnessed by improved static peels is achieved. Although, the hot melt adhesive can be semi-crystalline without the presence of wax, wax can be added to the composition in limited amounts to adjust other properties.

The hot melt adhesive composition can have a Heat of Fusion as tested according to the Heat of Fusion test method of 6.0 joules/gram, 6.5 joules/gram, 7 joules/gram, 7.5 joules/gram, 8 joules/gram to 13 joules/gram, 15 joules/gram, 18 joules/gram, 20 joules/gram, 25 joules/gram or any two values therebetween.

The hot melt adhesive composition can have a Dynamic Peel as tested according to the Dynamic Peel test method of at least 80 grams of force (gf)/3 inches (in), at least 120 gf/3 in, at least 160 gf/3 in, from 120 gf/3 in to 500 gf/3 in or even 300 gf/3 in to 500 gf/3 in.

The hot melt adhesive composition can have a Static Peel as tested according to the Static Peel test method of at least 5 minutes, at least 6 minutes, at least 7 minutes, at least 8 minutes, or even at least 10 minutes.

The hot melt adhesive composition can have a Brookfield Viscosity at 150° C. of no greater than 25,000 cP, no greater than 20,000 cP, no greater than 15,000 cP, from 250 cP to 30,000 cP, from 250 cP to 20,000 cP, or even from 500 cP to 15,000 cP

### Polymer

The hot melt adhesive composition includes two different propylene-based copolymers, a first propylene-based copolymer and a second propylene-based copolymer. The propylene-based copolymers are copolymers of propylene and at least one alpha-olefin.

Suitable propylene/alpha-olefin copolymers are derived from propylene and at least one alpha-olefin comonomer examples of which include alpha-olefin comonomers having from two to ten carbon atoms including, e.g., ethylene, butene, 1,4-methylpentene, hexene, octene, and combinations thereof. In one embodiment, at least one of the propylene-based copolymers, or even both propylene-based copolymers are propylene ethylene copolymers. The propylene-based copolymers can be based entirely on olefins, i.e., do not contain any functional groups. The propylene-based copolymers can include at least 75% by weight propylene or even at least 80% by weight propylene. The propylene-based copolymers can have a polydispersity (Mw/Mn) as tested by size exclusion chromatography (SEC) of less than 5, less than 3, less than 2.5, or even 2. Useful propylene-based copolymers include single-site (e.g., metallocene) catalyzed propylene-based polymers and Amorphous Poly Alpha Olefins (APAO's).

Both the first propylene-based copolymer and the second propylene based copolymer can have a melt index according to ASTM D1238 (230° C., 2.16 kgs) of from 1 grams/10 min, 2 grams/10 min, 3 grams/10 min, 4 grams/10 min, 5 grams/min, 6 grams/10 min to 13 grams/10 min, 15 grams/10 min, 20 grams/10 min, 30 grams/10 min, 40 grams/10 min, 50 grams/10 min, 75 grams/10 min or any two values there between.

The total amount of the first propylene-based copolymer and the second propylene-based copolymer can be from 10% by weight, 12% by weight, 13% by weight to 20% by weight, 22% by weight, 25% by weight, 30% by weight, 35% by weight, 40% by weight, or between any two values there between.

### First Propylene-based Copolymer

The first propylene-based copolymer can be a single site catalyzed polymer. The hot melt adhesive composition can include more than one first propylene-based copolymer.

The first propylene-based copolymer can be a single site catalyzed propylene ethylene copolymer. The first propylene-based copolymer has a comonomer content of from 0.5% by weight, 1% by weight, 1.5% by weight, 2% by weight to less than 5% by weight, less than 6% by weight, less than 7%, less than 8% by weight or any two values therebetween.

The first propylene-based copolymer has a melt index according to ASTM D1238 (230° C., 2.16 kgs) of from 1 grams/10 min, 2 grams/10 min, 3 grams/10 min, 4 grams/10 min, 5 grams/min, 6 grams/10 min to 13 grams/10 min, 15 grams/10 min, 20 grams/10 min, 30 grams/10 min, 35 grams/10 min, 40 grams/10 min, 45 grams/10 min, 50 grams/10 min, 75 grams/10 min or any two values there between.

The first propylene-based copolymer can have a Vicat Softening Temperature (as measured by the ExxonMobil test method) of at least 80° C., at least 85° C., at least 90° C., from 80° C. to 120° C., from 85° C. to 115° C., or even from 90° C. to 110° C. The inventors found that the inclusion of a propylene polymer with a relatively high Vicat Softening Temperature was helpful to achieve the desired cohesion.

The first propylene-based copolymer can have a Heat of Fusion as tested according to the Heat of Fusion test method of from 38 joules/gram, 40 joules/gram, 43 joules/gram, 45 joules/gram, 48 joules/gram, 50 joules/gram, 52 joules/gram, 55 joules/gram to 60 joules/gram, 62 joules/gram, 64 joules/gram, 65 joules/gram, 66 joules/gram, 68 joules/gram or any two values therebetween.

Useful commercially available first propylene-based copolymers include VISTAMAXX 3588 FL available from ExxonMobil Chemical (Houston, Texas).

The first propylene-based copolymer is present in the composition at from 2% by weight, 3% by weight, 4% by weight, 5% by weight, 6% by weight, 7% by weight to 12% by weight, 15% by weight, 18% by weight, 20% by weight, 25% by weight or any two values there between.

### Second Propylene-based Copolymer

The second propylene-based copolymer can be selected from a group consisting of single site catalyzed polymers and Zeigler-Natta catalyzed polymers (e.g., APAO polymers). The hot melt adhesive composition can include more than one second propylene-based copolymer. The second propylene-based copolymer can have a comonomer content of 5% by weight, 6% by weight, 7% by weight, 8% by weight, 9% by weight, 10% by weight to 16% by weight, 18% by weight, 20% by weight, 25% by weight, 30% by weight or any two values there between.

The second propylene-based copolymer can have a melt index according to ASTM D1238 (230° C., 2.16 kgs) of from 1 grams/10 min, 2 grams/10 min, 3 grams/10 min, 4 grams/10 min, 5 grams/min, 6 grams/10 min to 13 grams/10 min, 15 grams/10 min, 20 grams/10 min, 30 grams/10 min, 40 grams/min, 50 grams/10 min, 75 grams/10 min or any two values there between.

The second propylene-based copolymer can alternately have a Brookfield Viscosity at 190° C. of no greater than 25,000 cps, no greater than 20,000 cps, no greater than 15,000 cps, no greater than 12,000 cps, no greater than 10,000 cps, from 2,500 cps to 15,000 cps, or even from 3,000 cps to 12,000 cps.

The second propylene-based copolymer can have a Heat of Fusion as tested according to the Heat of Fusion test method of 0 joules/gram, 5 joules/gram, 7 joules/gram to 15 joules/gram, 20 joules/gram, 25 joules/gram, 30 joules/gram or any two values therebetween.

Useful commercially available second polymers include VISTAMAXX 8380 and VISTAMAXX 6202 (single site catalyzed propylene ethylene copolymer) commercially available from ExxonMobil Chemical (Houston, Texas) and AERAFIN 180 (APAO) and AERAFIN 75H (APAO) commercially available from Synthomer Adhesive Technology (Kingsport, Tennessee).

The second propylene-based copolymer is present in the composition at from 2% by weight, 3% by weight, 4% by weight, 5% by weight, 6% by weight, 7% by weight to 12% by weight, 15% by weight, 18% by weight, 20% by weight, 25% by weight, 30% by weight, 35% by weight, 40% by weight or any two values there between.

### Tackifying Agent

The hot melt adhesive composition includes a tackifying agent. The tackifying agent can be at least partially hydrogenated to improve the odor of the adhesive. The tackifying agent can be fluid or solid at room temperature. Suitable classes of tackifying agents include, e.g., aromatic, aliphatic and cycloaliphatic hydrocarbon resins, mixed aromatic and aliphatic modified hydrocarbon resins, aromatic modified aliphatic hydrocarbon resins, and hydrogenated versions thereof; terpenes, modified terpenes, and hydrogenated versions thereof; natural rosins, modified rosins, rosin esters, and hydrogenated versions thereof, and combinations thereof.

The tackifying agent can have a Ring & Ball Softening Point as reported by the supplier of from 80° C. to 150° C., from 90° C. to 140° C., from 90° C. to 135° C., from 90° C. to 130° C., or even from 90° C. to 110° C.

Useful tackifying agents are commercially available under a variety of trade designations including, e.g., the ESCOREZ series of trade designations from Exxon Mobil Chemical Company (Houston, Texas) including ESCOREZ 2203LC, ESCOREZ 5400 and ESCOREZ 5600, the EASTOTAC series of trade designations from Synthomer PLC including EASTOTAC H-100R and EASTOTAC H-100L, the RESINALL series of trade designations from Resinal Corp (Severn, NC) including RESINALL 1095S, the KOLON series of trade designations from Kolon Industries, Inc. (Ulsan, Korea) including SUKOREZ SU100 and SU110, the ARKON series of trade designations from Arakawa Chemical Industries, Ltd. (Osaka, Japan) including ARKON M90, ARKON M100 and ARKON P100, the WINGTACK series of trade designations from Cray Valley HSC (Exton, Pennsylvania) including WINGTACK 86, WINGTACK EXTRA, and WINGTACK 95, the PICCOTAC and KRISTALEX series of trade designations from Synthomer LLC including, e.g., PICCOTAC 8095 and KRISTALEX 3100.

The tackifying agent is present in the hot melt composition at from 25% by weight, from 30% by weight, from 35% by weight, from 40% by weight to 55% by weight, 60% by weight, 65% by weight or any two values there between.

### Plasticizer

The hot melt adhesive composition includes a plasticizer. Suitable plasticizers include, e.g., naphthenic oils, paraffinic oils (e.g., cycloparaffin oils), mineral oils, phthalate esters, adipate esters, olefin oligomers, polybutene, polyisoprene, hydrogenated polyisoprene, polybutadiene, benzoate esters, animal oil, plant oils e.g., castor oil, soybean oil, derivatives of oils, glycerol esters of fatty acids, and combinations thereof.

Useful commercially available plasticizers include CALSOL 5550 oil from Calumet Specialty Products Partners, LP (Indianapolis, Indiana), KAYDOL OIL from Sonneborn (Tarrytown New York) PARAPOL polybutene from Exxon Mobil Chemical Company (Houston, Texas), OPPANOL polyisobutylene from BASF (Ludwigsjhafen, Germany), KRYSTOL 550 mineral oil from Petrochem Carless Limited (Surrey, England) and PURETOL 35 mineral oil from Petro Canada Lubricants Inc. (Mississauga, Ontario).

In one embodiment, the plasticizer is a naphthenic oil. The plasticizer is present in the hot melt adhesive composition in an amount of at least 20% by weight, at least 22% by weight, at least 24% by weight, at least 25% by weight, or even from 15% by weight, 18% by weight, 20% by weight, 22% by weight, 24% by weight, 25% by weight to 32% by weight, 33% by weight, 34% by weight, 35% by weight, 36% by weight, 38% by weight, 40% by weight or any two values there between.

### Wax

The hot melt adhesive composition can be free of a wax, alternatively the hot melt adhesive composition can include a wax. Useful classes of wax include, e.g., paraffin waxes, microcrystalline waxes, high density low molecular weight polyethylene waxes, by-product polyethylene waxes, polypropylene waxes, Fischer-Tropsch waxes, oxidized Fischer-Tropsch waxes, functionalized waxes such as acid, anhydride, and hydroxyl modified waxes, animal waxes, vegetable waxes (e.g., soy wax) and combinations thereof. Useful waxes are solid at room temperature and preferably have a Ring and Ball softening point of from 50° C. to 170° C. The wax can be a propylene-based wax with a Mettler Softening Point (ASTM D 6092) of greater than 130° C., greater than 140° C., or even greater than 150° C. Useful waxes are commercially available from a variety of suppliers including EPOLENE N and C series of trade designations from Westlake Chemical Corporation (Houston, Texas) including e.g., EPOLENE N-21, EPOLENE N-15 and the LICOCENE series of trade designations from Clariant International Ltd. (Muttenz, Switzerland) including e.g., LICOCENE U.S. Plant Pat. No. 6,102, LICOCENE U.S. Plant Pat. No. 6,502 TP and LICOCENE U.S. Plant Pat. No. 7,502 TP.

The hot melt adhesive composition can include no greater than 6% by weight, no greater than 5% by weight, from 0.5% by weight to 6% by weight, from 0.5% by weight to 5% by weight, from 0.5% by weight to 4% by weight, or even from 0.5% by weight to 3% by weight of a wax.

### Additional Components

The hot melt adhesive composition optionally includes additional components including, e.g., stabilizers, antioxidants, additional polymers (e.g., styrenic block copolymers, polyethylene polymers e.g., single-site catalyzed ethylene-based polymers, additional propylene-based polymers), adhesion promoters, ultraviolet light stabilizers, corrosion inhibitors, colorants (e.g., pigments and dyes), fillers, surfactants, wetness indicators, superabsorbents, coatings, packaging films and combinations thereof.

Useful antioxidants include, e.g., pentaerythritol tetrakis [3, (3,5-di-tert-butyl-4-hydroxyphenyl)propionate], 2,2′-methylene bis (4-methyl-6-tert-butylphenol), phosphites including, e.g., tris-(p-nonylphenyl)-phosphite (TNPP) and bis (2,4-di-tert-butylphenyl) 4,4′-diphenylene-diphosphonite, di-stearyl-3,3′-thiodipropionate (DSTDP), and combinations thereof. Useful antioxidants are commercially available under a variety of trade designations including, e.g., the IRGANOX series of trade designations including, e.g., IRGANOX 1010, IRGANOX 565, and IRGANOX 1076 hindered phenolic antioxidants and IRGAFOS 168 phosphite antioxidant, all of which are available from BASF Corporation (Florham Park, New Jersey), and ETHYL 702 4,4′-methylene bis (2,6-di-tert-butylphenol). When present, the adhesive composition preferably includes from 0.1% by weight to 2% by weight antioxidant.

### Disposable Absorbent Article

The hot melt adhesive composition can be applied to (i.e. such that it is in direct contact with) or incorporated in a variety of substrates within the disposable absorbent article including, e.g., films (e.g., polyolefin (e.g., polyethylene and polypropylene) films), release liners, porous substrates, cellulose substrates, sheets (e.g., paper, and fiber sheets), paper products, woven and nonwoven webs, fibers (e.g., synthetic polymer fibers and cellulose fibers), elastics and tape backings.

The hot melt adhesive composition is also useful in a variety of applications and constructions including, e.g., disposable absorbent articles including, e.g., disposable diapers, adult incontinence products, sanitary napkins, medical dressings (e.g., wound care products), bandages, surgical pads, pet training pads (e.g. puppy pads) and meat-packing products, and components of absorbent articles including, e.g., an absorbent element, absorbent cores, impermeable layers (e.g., backsheets), tissue (e.g., wrapping tissue), acquisition layers and woven and nonwoven web layers (e.g., top sheets, absorbent tissue).

The hot melt adhesive composition is useful on substrates made from a variety of fibers including, e.g., natural cellulose fibers such as wood pulp, cotton, silk and wool; synthetic fibers such as nylon, rayon, polyesters, acrylics, polypropylenes, polyethylene, polyvinyl chloride, polyurethane, and glass; recycled fibers, and various combinations thereof.

Various application techniques can be used to apply the hot melt adhesive composition to a substrate including, e.g., slot coating, spraying including, e.g., spiral spraying and random spraying, screen printing, foaming, engraved roller, extrusion and meltblown application techniques.

### Methods of Making a Disposable Absorbent Article

The hot melt adhesive composition has good cohesive strength. The hot melt adhesive composition maintains this strength at a low coat weight. These properties make it useful in the construction of disposable absorbent articles, core stabilization and for elastic attachment.

The hot melt adhesive composition can be used for construction applications. In a typical construction application in the manufacture of a disposable absorbent article, a body fluid impermeable backsheet is bonded to a nonwoven substrate. The hot melt adhesive composition may also be used to bond at least one additional layer or material selected from the group consisting of absorbents, tissues, elastomeric materials, superabsorbent polymers, and combinations thereof. For example, the adhesive can further be used for back sheet lamination i.e., where the body fluid impermeable backsheet typically a polyolefin film (e.g., polyethylene, polypropylene, ethylene vinyl acetate, ethylene copolymer, etc.) is bonded to a second nonwoven to improve the feel of the disposable article.

The hot melt adhesive can also be used to contain and/or provide strength to the absorbent core of a disposable absorbent article (i.e., as a core stabilization adhesive). The absorbent core can include many different materials including natural cellulose fibers (e.g., wood pulp, fibers, cotton, fluff, etc.) and superabsorbent polymers (e.g., polyacrylates).

The hot melt adhesive composition can be used for elastic attachment applications, which include bonding elastic material (e.g., strands, film, etc.) to a component of the disposable absorbent article. The elastic material can be coated with the hot melt adhesive composition while in a stretched form and then bonded to a film (e.g., polyethylene, polypropylene, etc.) or a nonwoven substrate. The elastic material coated with the hot melt adhesive can also be bonded between two such substrates. This process results in creep resistant gathers once the tension is removed.

The adhesive can alternatively be applied to the film or nonwoven substrate and then the elastic material bonded to it. The hot melt adhesive can be used as an elastic attachment adhesive in forming the ear/tab of the disposable absorbent article. Alternatively, the hot melt adhesive can further be used as an elastic attachment adhesive to help elasticize either the leg holes or the waist of the disposable absorbent article.

The invention will now be described by way of the following non-limiting examples. All parts, ratios, percentages, and amounts stated in the Examples are by weight unless otherwise specified.

### EXAMPLES

### Test Procedures

Test procedures used in the examples and throughout the specification, unless stated otherwise, include the following.

### Viscosity Test Method

Viscosity is determined in accordance with ASTM D-3236 entitled, “Standard Test Method for Apparent viscosity of Adhesives and Coating Materials,” (Oct. 31, 1988), using a Brookfield Thermoset viscometer Model RVDV 2 and a number 27 spindle. The results are reported in centipoise (cps).

Heat of Fusion by Dynamic Scanning calorimetry

DSC measurements were performed on a Mettler Toledo DSC 3. Samples were evaluated in hermetically sealed aluminum pans. Sample sizes ranged from 9-12 mg. Each adhesive sample was conditioned for 10 minutes at 180° C. The sample was then cooled at 20° C./min and aged at 25° C. for 10 minutes. Following aging, the sample was cooled rapidly to-50° C. and ramped at 10° C./min until a temperature of 200° C. was reached. The Heat of Fusion values (reported here in J/g) were taken from this second heating ramp.

### Peel Test Sample Preparation

A slot coating applicator, which is 3 inch (in) (76.2 millimeter (mm)) wide, and a laminator are set to an application temperature of 157° C., without a bonding roll or end seal unit, using an application coat weight of 3 grams (g)/square meter (m2), and minimal rewind and unwind tensions so as not to stretch the web.

The hot melt adhesive composition is applied continuously on a polypropylene nonwoven web having a thickness of 0.1 mm and a basis weight of 15 g/m2. The nonwoven web is passed through the applicator at a speed of from 190 meters per minute (m/min) to 250 m/min with 5 mm deflection. A non-breathable, layered polyethylene film having a thickness of 0.23 mm, traveling at the same speed as the nonwoven web, is then adhered into place against the adhesive composition and the nonwoven web to form a laminate. The time between coating the adhesive to the nonwoven and adhering to the polyethylene film is 0.3-0.4 seconds.

### Dynamic Peel Adhesion Test Method

Dynamic Peel is determined according to ASTM D1876-01 entitled, “Test Method for Determining Peel Resistance of Adhesive (T-Peel Test Method),” with the exception that 8 replicates are run. The test samples are run on an IMASS Spec-type test instrument. The test samples are prepared as described in the Peel Test Sample Preparation Method. Eight test samples are prepared for each sample composition and the test samples are tested 24 hours after the test sample has been prepared. The average peel value over 10 seconds of peeling is recorded, and the results are reported in units of grams of force per 3 inches (gf/3 in).

### Static Peel Adhesion Test Method

Static Peel is determined by hanging a 150 g weight from a 2-inch-wide laminate in the cross direction (e.g., perpendicular to the direction of the web path of the laminator). The samples are tested in a controlled temperature and humidity room set to 25° C. and 50% relative humidity. The hang time of the weight is measured in minutes. Eight test samples are prepared for each sample composition and the test samples are tested 24 hours after the test sample has been prepared. The average hang time is reported.

TABLE 1
Polymer Properties
NA (Not Available)

| | VISTA- | VISTA- | VISTA- | VISTA- | VISTA- | VISTA- | VISTA- |
|---|---|---|---|---|---|---|---|
| | MAXX | MAXX | MAXX | MAXX | MAXX | MAXX | MAXX |
| | 6102 | 6502 | 6202 | 3000 | 3980FL | 3588FL | 8380 |
| Polymer Type | PP/PE | PP/PE | PP/PE | PP/PE | PP/PE | PP/PE | PP/PE |
| Ethylene content | 16 | 13 | 15 | 11 | 9 | 4 | 12 |
| (wt. %) | | | | | | | |
| Density (g/cm³) | 0.862 | 0.865 | 0.863 | 0.873 | 0.878 | 0.889 | 0.864 |
| Melt Index | 3 | 45 | 20 | 8 | 8 | 8 | NA |
| 230° C./2.16 kg | | | | | | | |
| (g/10 min) | | | | | | | |
| Heat of Fusion | NA | NA | 9.2 | NA | 36.8 | 58.0 | 18.5 |
| (j/g) | | | | | | | |
| Brookfield | NA | NA | NA | NA | NA | NA | 7,570 |
| Viscosity @ | | | | | | | |
| 190° C. (cps) | | | | | | | |
| Vicat Softening | 53.9 | 51.4 | 45.2 | 65.1 | 77.3 | 103 | NA |
| Temperature (as | | | | | | | |
| measured by the | | | | | | | |
| ExxonMobil test | | | | | | | |
| methods) (° C.) | | | | | | | |

TABLE 2
Examples
NT (Not tested)

| | Control | | | | | | | | |
|---|---|---|---|---|---|---|---|---|---|
| | 1 | | | | | | | | |
| | SBC | | | | | | | | |
| | Based | Ex 1 | Ex 2 | Ex 3 | Ex 4 | Ex 5 | Ex 6 | Ex 7 | Ex 8 |
| VISTAMAXX | | 19 | 19 | 19 | | 15 | | 19 | 19 |
| 8380 | | | | | | | | | |
| VISTAMAXX | | | | | 6 | | 6.5 | | |
| 6202 | | | | | | | | | |
| VISTAMAXX | | 9 | 9 | 9 | 8 | 10 | 8.5 | 9 | 9 |
| 3588FL | | | | | | | | | |
| ESCOREZ 5400 | | | | 42 | 54.75 | 43.75 | 50.75 | | |
| ESCOREZ 5415 | | | | | | | | 42 | |
| HENGHE | | | | | | | | | 42 |
| H51001 | | | | | | | | | |
| EASTOTAC | | 42 | 42 | | | | | | |
| H130W | | | | | | | | | |
| CALSOL 5550 | | 27.5 | 25.5 | 27.5 | 29 | 29 | 32 | 27.5 | 27.5 |
| EPOLENE N21 | | | 2 | | | | | | |
| IRGANOX 1010 | | 0.5 | 0.5 | 0.5 | 0.5 | 0.5 | 0.5 | 0.5 | 0.5 |
| COEXTRUSION | | 1.5 | 1.5 | 1.5 | 1.5 | 1.5 | 1.5 | 1.5 | 1.5 |
| COATING | | | | | | | | | |
| ZP FILM | | 0.5 | 0.5 | 0.5 | 0.25 | 0.25 | 0.25 | 0.5 | 0.5 |
| Brookfield | | 10600 | 16725 | 13100 | NT | 11430 | 12750 | 12360 | 10380 |
| Viscosity @ | | | | | | | | | |
| 275° F. (cP) | | | | | | | | | |
| Brookfield | | 6225 | 7813 | 7750 | 5900 | 6688 | 6876 | 7250 | 5463 |
| Viscosity @ | | | | | | | | | |
| 300° F. (cP) | | | | | | | | | |
| Brookfield | | 3990 | 5275 | 5013 | 3580 | 4250 | 4318 | 4547 | 3863 |
| Viscosity @ | | | | | | | | | |
| 325° F. (cP) | | | | | | | | | |
| Brookfield | | 2660 | NT | 3295 | 2360 | NT | NA. | 3132 | 2570 |
| Viscosity @ | | | | | | | | | |
| 350° F. (cP) | | | | | | | | | |
| Dynamic Peel | 79 | 80 | NT | 377 | 268 | 573 | 488 | 163 | 324 |
| (gf/3 in) | | | | | | | | | |
| Static Peel (min) | 4.6 | 7.7 | NT | 15.0 | 50.5 | 6.2 | 15.1 | 6.6 | 7.4 |
| Delta H fusion | 0 | 9.2 | 17.0 | 10.0 | 8.5 | 11.0 | 8.0 | 9.4 | 11.0 |
| (J/g) | | | | | | | | | |

Other embodiments are within the claims.

What is claimed is:

1. A hot melt adhesive composition comprising:
a. a first propylene-based copolymer that has a comonomer content of less than 5% by weight, a Heat of Fusion from 40 joules/gram to 65 joules/gram and a Melt Flow Rate of from 2 grams/10 minute to 40 grams/10 minute when tested at 230° C./2.16 kg according to ASTM D 1238,
b. a second propylene-based copolymer that has a comonomer content of from 5% by weight to 30% by weight,
c. a tackifying agent; and
d. a plasticizer.

2. The hot melt adhesive composition of claim 1 wherein the first propylene-based copolymer and the second propylene-based copolymer are metallocene catalyzed.

3. The hot melt adhesive composition of claim 1 wherein the second propylene-based copolymer has a Brookfield Viscosity at 190° C. of no greater than 20,000 cps.

4. The hot melt adhesive composition of claim 1 comprising at least 22% by weight of a plasticizer.

5. The hot melt adhesive composition of claim 1 comprising:
a. from 4% by weight to 20% by weight of the first propylene-based copolymer,
b. from 4% by weight to 25% by weight of the second propylene-based copolymer,
c. from 30% by weight to 60% by weight of the tackifying agent, and
d. from 22% by weight to 35% by weight of the plasticizer.

6. A hot melt adhesive composition of claim 1 wherein the second propylene-based copolymer that has a comonomer content of from 8% by weight to 30% by weight and a Melt Flow Rate of from 2 grams/10 minute to 50 grams/10 minute when tested at 230° C./2.16 kg according to ASTM D 1238.

7. The hot melt adhesive composition of claim 1 wherein the total of the first propylene-based copolymer and the second propylene-based copolymer makes up from 10% by weight to 20% by weight of the hot melt adhesive composition.

8. The hot melt adhesive composition of claim 1 wherein the first propylene-based copolymer has a melt flow rate of from 2 grams/10 minute to 20 grams/10 minute when tested at 230° C./2.16 kg according to ASTM D 1238.

9. The hot melt adhesive composition of claim 1 wherein the first propylene-based copolymer has a Heat of Fusion of from 45 joules/gram to 65 joules/gram.

10. The hot melt adhesive composition of claim 1 wherein the first propylene-based copolymer and second propylene-based copolymer are propylene ethylene copolymers.

11. The hot melt adhesive composition of claim 1 wherein the tackifying agent is a hydrogenated hydrocarbon.

12. The hot melt adhesive composition of claim 1 wherein the tackifying agent has a Ring & Ball Softening Point of from 90° C. to 110° C.

13. The hot melt adhesive composition of claim 1 wherein the plasticizer is an oil.

14. The hot melt adhesive composition of claim 1 further comprising a wax.

15. The hot melt adhesive composition of claim 1 having a Heat of Fusion of from 6 joules/gram to 20 joules/gram.

16. A disposable absorbent article comprising the hot melt adhesive composition claim 1.

17. A disposable absorbent article comprising:
a. a first substrate,
b. a second substrate, and
c. the hot melt adhesive composition of claim 1,
d. wherein the hot melt adhesive composition is applied to at least one of the first and second substrate.

18. The disposable absorbent article of claim 17 selected from the group consisting of diaper, adult incontinence article and sanitary hygiene article.

19. The disposable absorbent article of claim 17 wherein the hot melt adhesive composition is used in the disposable absorbent article for an application selected from the group consisting of construction and back sheet lamination.

20. A hot melt adhesive composition comprising:
a. from 4% by weight to 20% by weight of a first propylene-based copolymer that is single-site catalyzed, has a comonomer content of less than 5% by weight, a Heat of Fusion from 40 joules/gram to 65 joules/gram and a Melt Flow Rate of from 2 grams/10 minute to 40 grams/10 minute when tested at 230° C./2.16 kg according to ASTM D 1238,
b. from 4% by weight to 25% by weight of a second propylene-based copolymer that has a comonomer content of from 5% by weight to 30% by weight,
c. from 30% by weight to 60% by weight of a tackifying agent and
d. from 22% by weight to 35% by weight of a plasticizer.
